(19) United States
(12) Plant Patent Application Publication
(10) Pub. No.: US 2014/0173795 P1
(43) Pub. Date: Jun. 19, 2014

(54) Medinilla hybrid named 'MAGIC'

(50) ;

(71) Applicant: CORN.BAK B.V., Assendelft (NL)

(72) Inventors: Elly Bak, Rijsenhout (NL); Nicolaas D.M. Steur, Oude Niedorp (NL)

(73) Assignee: CORN.BAK B.V., Assendelft (NL)

(21) Appl. No.: 13/694,587

(22) Filed: Dec. 14, 2012

(51) Int. Cl. A01H 5/00

(52) U.S. Cl. PLT/263.1

(57) ABSTRACT

A new and distinct Medinilla sp. plant named ‘MAGIC’, characterized by a erect shrub plant, measuring about 25-30 cm in total height (when first flowering) and about 30 cm in diameter; green colored leaves, measuring about 12 cm in length and about 3.5-4.5 cm in width; and pendulous panicles, measuring about 20 cm in length and about 10 cm in diameter, with red (RHS 53C) flowers.

### LATIN NAME OF THE GENUS AND SPECIES OF THE PLANT CLAIMED

Medinilla sp.

### VARIETY DENOMINATION

‘MAGIC’

### BACKGROUND OF THE INVENTION

The present invention relates to a new and distinct hybrid cultivar of Medinilla plant, botanically known as Medinilla sp., hereinafter referred to by the cultivar name ‘MAGIC’. The genus of Medinilla belongs to the Melastomataceae family. The new Medinilla ‘MAGIC’ is a product of a planned breeding program conducted by the inventors, Elly Bak and Nicolaas D.M. Steur, in Assendelft, The Netherlands. The objective of the breeding program is to develop a new Medinilla variety with good growing habit and attractive inflorescence. The new Medinilla ‘MAGIC’ originated from a cross made in a controlled breeding program by the inventors in 2008 in Assendelft, The Netherlands. The female or seed parent is the Medinilla selection designated ‘84068295’ (unpatented), and the male or pollen parent is the Medinilla selection designated ‘84064514’ (unpatented). ‘MAGIC’ was discovered and selected by the inventors in 2010, as a single flowering plant within the progeny of the stated cross in a controlled environment in Assendelft, The Netherlands.

Asexual reproduction of the new Medinilla hybrid ‘MAGIC’ performed by vegetative means by cuttings was first performed in 2010, in Assendelft, The Netherlands. The first ‘MAGIC’ plants propagated through the use of cuttings flowered in 2011, in Assendelft, The Netherlands, and have demonstrated that the combination of characteristics as herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be unique characteristics of ‘MAGIC’ which in combination distinguish this Medinilla hybrid as a new and distinct cultivar:

1. Solid growth habit;

2. Erect shrub plant, measuring about 25-30 cm in height (above the pot when first flowering);

3. Green colored leaves, measuring about 12 cm in length and about 4 em in width;

4. Pendulous panicles with red (RHS 53C) colored flowers;

Of the many commercial cultivars known to the present inventors, the most similar in comparison to the new Medinilla variety ‘MAGIC’ is the Medinilla variety ‘BELLA’ (U.S. Plant patent application Ser. No. 13/067,309). Plants of the new Medinilla ‘MAGIC’ differ from plants of Medinilla ‘BELLA’ primarily in leaf size, bract size, and inflorescence color. Plants of ‘MAGIC’ have smaller leaves and bracts than plants of ‘BELLA’. Additionally, plants of ‘MAGIC’ have more inflorescences.

### BRIEF DESCRIPTION OF THE PHOTOGRAPHS

The accompanying photographs illustrate the overall appearance of the new Medinilla ‘MAGIC’ showing the colors as true as is reasonably possible with colored reproductions of this type. Colors in the photographs may differ slightly from the color values cited in the detailed botanical description which accurately describes the color of ‘MAGIC’.

FIG. 1 shows a side view perspective of a typical potted flowering plant of ‘MAGIC’ at about 50 weeks from potting.

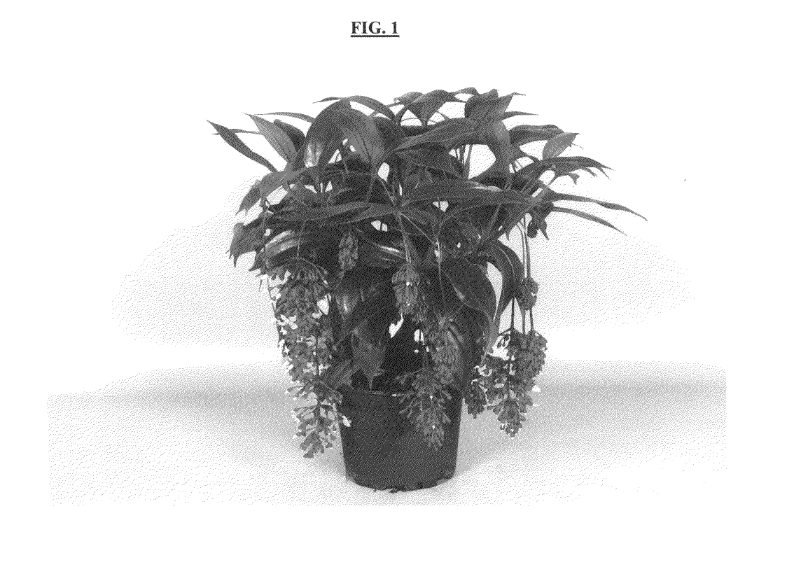

FIG. 2 shows a close up view of a typical panicle of ‘MAGIC’ on a flowering plant at about 50 weeks from potting.

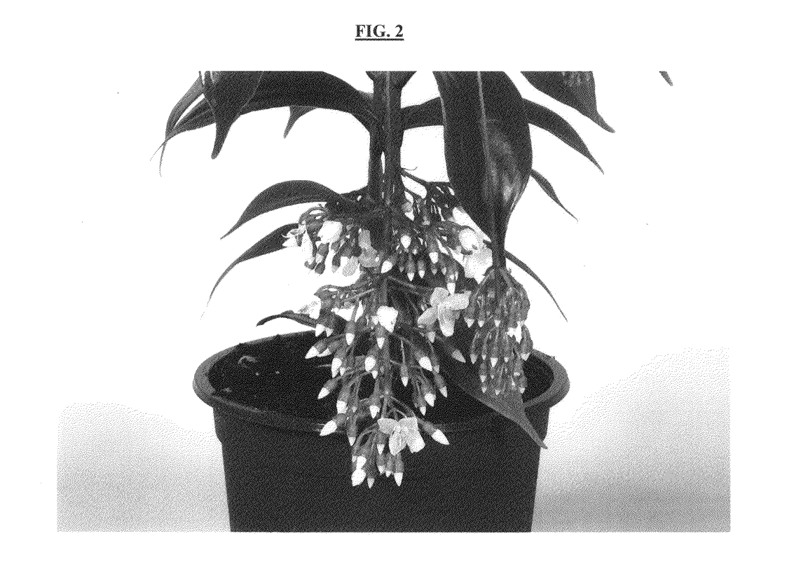

### DETAILED BOTANICAL DESCRIPTION

‘MAGIC’ has not been observed under all possible environmental conditions. The phenotype of the new cultivar may vary with variations in environment such as temperature, light intensity, day length, fertilizers and humidity without any change in the genotype of the plant. The aforementioned photograph, together with the following observations, measurements and values describe the new Medinilla ‘MAGIC’ as grown in a greenhouse in Assendelft, The Netherlands, under conditions which closely approximate those generally used in commercial practice.

Color references are made to The Royal Horticultural Society Colour Chart (R.H.S.), 2001 Edition, except where general colors of ordinary significance are used. Color values were taken under daylight conditions in a greenhouse in Assendelft, The Netherlands. The age of the plants of ‘MAGIC’ described is about 50 weeks from potting.
Classification:
   Botanical.—Medinilla sp.
Parentage:
   Female parent.—Medinilla selection designated ‘84068295’ (unpatented).
   Male parent.—Medinilla selection designated ‘84064514’ (unpatented).
Propagation: Cuttings
Plant:
   General appearance and form.—Height: About 25-30 cm Diameter: About 30 cm Shape: Erect shrub. Growth habit: stem Plant Vigor: good Flowering Season: from April until October Cold Tolerance: frost tender, temperatures below 5C may damage plants.
   Fragrance.—none.
Foliage:
   Quantity.—About 40.
   Size.—Length: About 12 cm. Width: About 3.5-4.5 cm.
   Overall shape.—elliptical.
   Apex shape.—cuspidate.
   Base shape.—acute.
   Margin.—slightly undulate.
   Texture.—smooth.
   Color.—Leaf color can vary somewhat depending on growing conditions. Upper Surface: Glossy green, ranges between RHS 137A and RHS 139A Lower Surface: Green, ranges between RHS 137C and RHS 143A.
   Venation.—sunken.
Inflorescence:
   Shape.—Pendulous panicles.
   Amount.—about 20 (depending on the size of the plant).
   Size.—Length: About 20 cm Width: About 10 cm.
   Color.—red, closest to RHS 53C.
   Corolla.—Number: about 120 per panicle.
   Petals.—Number: 4 or 5 per flower Length: 10 mm Width: 7 mm Overall Shape: obovate, asymmetrical Apex Shape: obtuse Color: Upper surface: white, closest to RHS 155D Lower surface: white, closest to RHS 155D.
   Sepals.—growth together, enclosing the ovary. Color: red, closest to RHS 53C.
   Bracts.—Quantity: 4 per crown Arrangement: verticillate.
   Size.—Length: about 1-13 mm Width: less than 1-2 mm.
   Overall shape.—elliptical.
Reproductive organs:
Androecium:
   Stamen.—Number: 8-10 per flower Length: 7 mm Diameter: less than 1 mm Color: light yellow, with a white base (too small to qualify R.H.S. value).
   Anther.—Length: 8 mm Color: violet, closest to RHS 87B, with a yellow base (too small to qualify R.H.S. value) Pollen: not visible.
Gynoecium:
   Pistil.—Number: 1 per flower Length: 12 min.
   Stigma.—Width: less than 1 mm Color: white, too small to qualify R.H.S. value.
   Style.—Length: 17 mm Color: white, closest to RHS 155B.
   Ovary.—Shape: round Position: inferior Length: 3 mm Diameter: 3 mm Color: white, closest to RHS 155C.
Seeds: No seeds produced
Fruit: No fruit produced
Disease/pest resistance: No observations made
Disease/pest susceptibility: No observations made

What is claimed is:

1. A new and distinct Medinilla plant named ‘MAGIC’, substantially as illustrated and described herein.
